(19) United States
(12) Patent Application Publication
(10) Pub. No.: US 2007/0053997 A1
(43) Pub. Date: Mar. 8, 2007

(54) Thermal herbal applicator

(76) Inventor: Chor Ee Ewe, Penang (MY)

Correspondence Address: DONN K. HARMS;PATENT & TRADEMARK LAW CENTER, SUITE 100, 12702 VIA CORTINA, DEL MAR, CA, 92014, US

(21) Appl. No.: 10/571,749

(22) PCT Filed: Sep. 2, 2004
(86) PCT No.: PCT/SG04/00277

§ 371 Date: Mar. 13, 2006

(30) Foreign Application Priority Data
Sep. 18, 2003 (MY) PI 2003 3564

(51) Int. Cl. A61K 36/00; H05B 1/00

(52) U.S. Cl. 424/725; 219/201

(57) ABSTRACT

A herbal applicator that comprises of a flexible Main Housing which can be easily attached on to the body, is affixed with a flexible Heater Pad that connects to an electrical Power Source to, be regulated by an adjustable Temperature Controller, with the said flexible Heater Pad resting on a Herbal Pack which is in turn retained by a flexible Herbal Pack Retainer made of a permeable material and built in such a way as to allow easy removal of the Herbal Pack for replenishment.

### FIELD OF THE INVENTION

The invention relates generally to a herbal applicator and more specifically to a thermal herbal applicator using transdermal transfer.

### BACKGROUND OF THE INVENTION

Medicinal herbs are some of our oldest medicines, and their increasing use in recent years is evidence of the strong public interest in having alternatives to conventional medicine.

There are various methods of using herbs as a cure to an ailment and one of the most effective methods often practiced is by the application of heat onto the herbs so as to fully vapourize the said herbs in order that they may be absorbed through the skin into the tissues of the body or blood, and thereby effectively acting on the cells of the human body to provide a cure or to improve the immune mechanisms of the body. These herbal vaporizers uses heat to release the herbal essence of any herbal material at a temperature below that where it combusts, as the active ingredient (herbal essence) burns at a far lower temperature of the substance material (fiber) around it Thus the essential ingredient may be released into a fie vapour mist without burning the substance material. Therefore the vaporizer rids of the harmful toxin smoke aerosol, carbon monoxide and carcinogenic elements created by burning of substance material.

Applicators using this concept where the trapped herbal essence between the Herbal Pack and the body skin causes a surge or infusion of herbal essence deep into the skin, result in enhancing peripheral circulation; activating texture cells, strengthening physiological mechanism; hastening immunity system easing chronic diseases, proving the metabolism of Lactic Acid; relieving joints and muscles pain and promoting tissue repairing and natural restorative processes.

These forms of thermal herbal applicators are well documented and illustrated in moxibustion treatment, and are found in a number of prior art patents, like U.S. Pat. No. 4,090,517 entitled “Medical Appliance”; U.S. Pat. No. 4,747,841 entitled. “Methods and Instrument of Moxibustion”, wherein is described a method of and apparatus for moxibustion comprising of feeding air to a heat generating composition in contact with a herb material comprising moxa, such that said heat generating composition comprises a pyrogen and said herb material is located adjacent to a skin surface, wherein said feeding causes said pyrogen to generate heat by oxidation, whereby said herb material is heated and vapourized and the generated heat and vapour act on the skin, causing moxibustion effect.

Such prior art inventions establish the moxibustion effect which essentially is a transdermal transfer of a herbal material which is being heated till it vapourizes and the resultant generated heat and vapour combines to act on the skin.

However, here, in its original form, the heat is generated by oxidation rather than from an external source which could also provide temperature regulation at the same time, besides there are no means for replenishment and the herbal sachets or pads cannot be changed when required.

A more recent example is found in U.S. Pat. No. 6,253,104 entitled “Method of preparing Pharmaceutical Moxa extract and apparatus for electrical moxibustion using the same extract” Here the applicator comprises of a heating means drawn from an electrical supply, a fixing means for fixing and supporting the heating means, adiabatic means for preventing the heat from dispersing to the upper side directions and a herbal pad means for containing the herbs.

After injecting the pharmaceutical moxa extract formulation into a liquid permeable membrane, a moxibustion pad can retain the liquid permeable membrane having the pharmaceutical moxa extract formulation, and in which moxibustion treatment can be easily administered by attaching the skin electrode type electrical moxibustion apparatus on the affected parts and heated indirectly by an electrical heater such that the crude drag components of the moxa can be naturally permeated into the skin.

However, this invention does not cater for natural herbs in powder form for it only caters for extract lotion or tincture.

However, the use of traditional natural herbs, besides moxa or a moxa extract are now encouraged and are found to be equally effective if not more effective, especially when the natural herbs are prepared in powder form which would give rise to certain advantages and widen possibilities not hereto realized or encountered in the prior art applicators, which would include e.g. less expensive, faster processing, more economical to use and more user friendly applications. However, there is undeniable evidence to suggest that it is often the whole, unrefined plant (natural, raw material) as utilized by traditional Chinese medical practitioners that may be more effective than a particular substance extracted for it and used in isolation.

In present day herbal therapy, it is also necessary to be able to administer the herbs at the correct temperature; using different combination of herbs on different applications; and ability to replenish or change the herbs easily as when or where necessary Prior art applicators do not cater for such versatility, and there is an urgent need for an improved thermal herbal applicator that can provide for the aforesaid functions.

### SUMMARY OF THE INVENTION

It is the object of the present invention to provide for such a simple, versatile, easily adaptable and economical device that can cater for the use of other forms of herbs, especially in their natural, state instead of extracts, to incorporate the use of heat on the said herbs, so as to vapourize the said herbs, until they can be easily permeated into the skin of the person, when the said applicator is applied and attached to the skin of the particular person.

It is another object of the invention that the temperature of the herbs can be regulated to suit the particular type of herbs or the therapy involved.

It is another object of the invention to provide for an applicator where the herbs may be easily replenished or changed when required.

It is a further object of the present invention to provide an adjustable and flexible thermal herbal applicator that can be easily attached on to the body and following the contours of the body so as to effectively administer, the heated herbs directly to the skin

To achieve the foregoing objectives and in accordance with the invention as embodied and, broadly described herein, the present invention comprises of a flexible main housing to trap the heat within, preferably made up of two sections (i) a top member fixed with a strap retainer means for attaching on to a strap means for strapping on to the body. (ii) a bottom member for holding the flexible flat heater pad affixed, to an attachment means, preferably a Velcro tape that connects to a corresponding Velcro tape that affixed on to a herbal pack retainer which in turn helps to hold the herbal pack in place, so that the same may be easily removed and replenished with a new herbal pack as and when desired. The heater pad is connected by a cable to an electrical power source wherein its temperature can be controlled by an electrical controller means.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanied drawings, wherein:

FIG. 1 is a plan, schematic view of the invention

FIG. 2 is an exploded perspective view of the invention

FIG. 3 is a cross sectional view of the invention

FIG. 4 shows the process of how the infusion of herbal essence penetrates deep into the skin.

FIG. 5 is a perspective view of the invention in its assembled form

FIG. 6 shows how the invention is applied on the skin of a specific body part.

### DETAILED DESCRIPTION OF A PREFERRED OF THE INVENTION

The features and other details of the method and apparatus of the invention will now be more particularly described with reference to the accompanying drawings and pointed out in the claims. The same numeral present in different figures represent the same item. It will be understood that the particular embodiments of the invention are shown by way of illustration and not as a limitation of the invention. The principal features of this invention can be employed in various embodiments without departing from the scope of this invention.

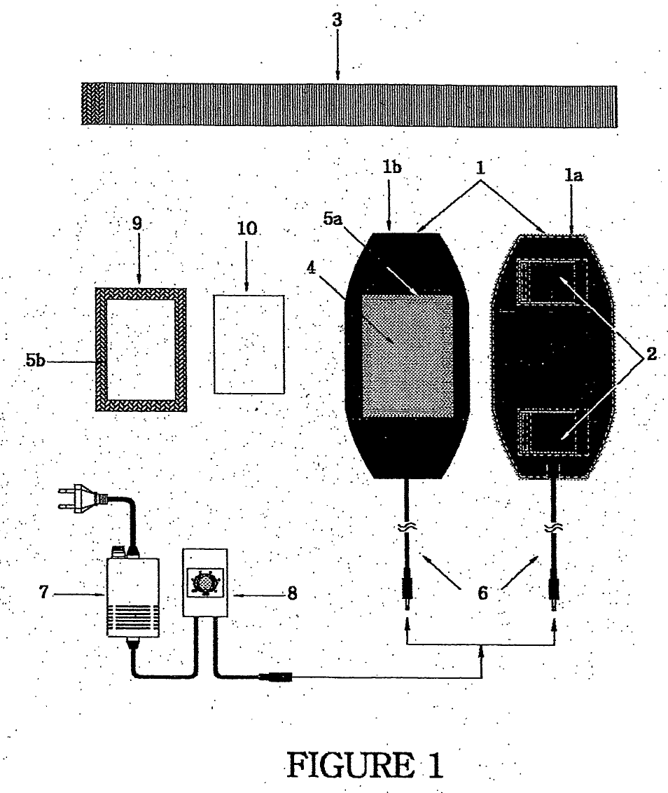

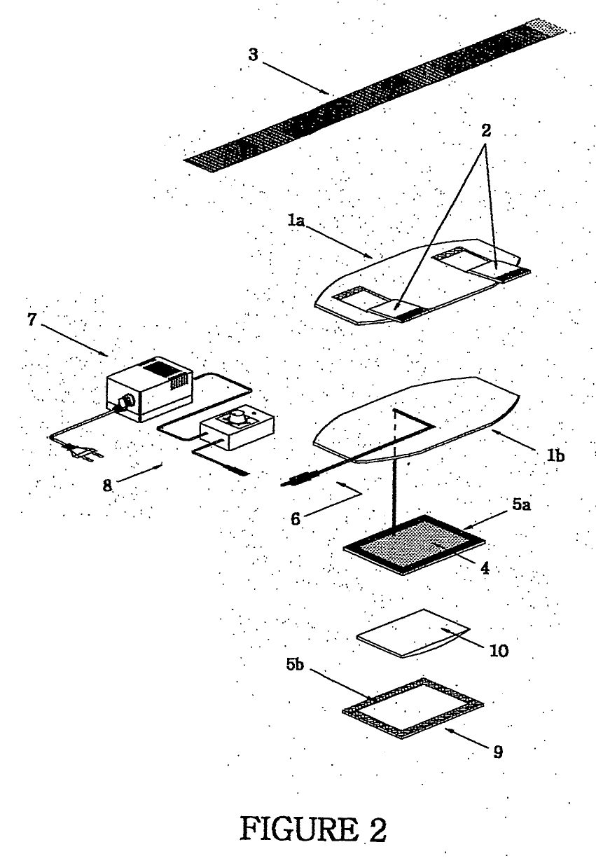

This invention has a structure that comprises of the following parts as shown in FIG. 1 and FIG. 2:

(i) a flexible Main Housing (1) for better retainer of heat made of flexible material formed with two sections:

(a) a top flexible Main Housing Member (1a) with a Strap Retainer Means (2) for attaching to a Strap Means (3) for strapping on to a part of the body.

(b) a bottom, flexible Main Housing Member (1b) with a flexible flat Heater Pad (4) for generating the heat, positioned at its bottom and affixed with an Attachment Means (5a) e.g. a Velcro tape, The flexible flat Heater Pad (4) is joined by an electrical Cable (6) which connects to an electrical Power Source (7) that is controlled by a Temperature Regulator (8) for regulating the required temperature,

(ii) a flexible Herbal Pack Retainer (9) which is affixed with a corresponding attachment means (5b) e.g. a velcro tape that is to be connected on to the attachment means (5a) that is affixed on the Heater Pad (4),

(iii) a Herbal Pack (10) for containing the herb or herbs, is to be strapped in between the Heater Pad (4) and a Herbal Pack Retainer (9) and held on by the Attachment Means (5a, 5b) so that the flexible Herbal Pack Retainer (9) which is to hold the Herbal Pack (10) in place resulting in such a way that the Herbal Pack (10) is in direct contact with thee Heater Pad (4) on one side and the Herbal Pack Retainer (9) on the other side, such that the Herbal Pack Retainer (9) is in direct contact with the skin of the body, when the apparatus is in use. The Herbal Pack Retainer (9) also serves to, absorb part of the heat and helps to reduce the vapourizing temperature to a curie temperature and, at the same time is made of permeable material to allow the herbal essence to permeate into the skin.

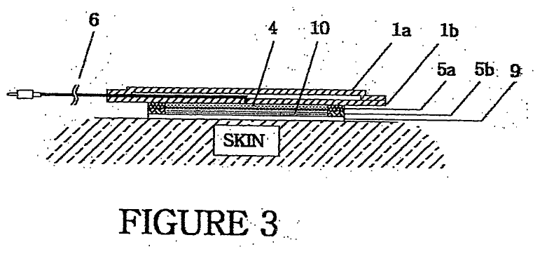

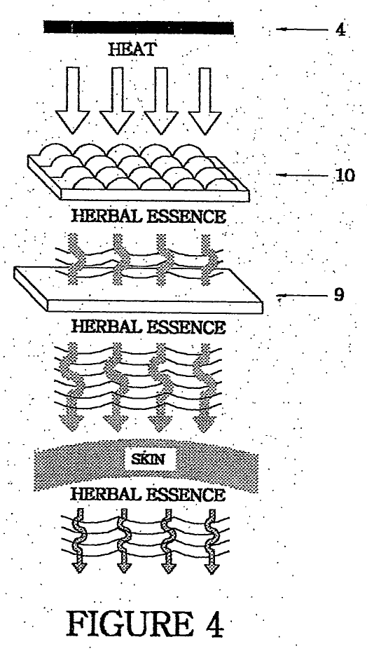

The entire assembly is as shown in FIG. 2. which is an exploded perspective view of the present invention with all the parts arranged in order of assembly. FIG. 3 shows the cross section of the invention with the relevant parts arranged in order of assembly and in contact with the skin of the body. The heat general applied on the herbs contained in, the Herbal Pack (10) would release the herbal essence which would then permeate through the thin membrane of the Herbal Pack Retainer (9) and thereby cause an infusion of herbal essence penetrating deep into the skin as illustrated clearly in FIG. 4.

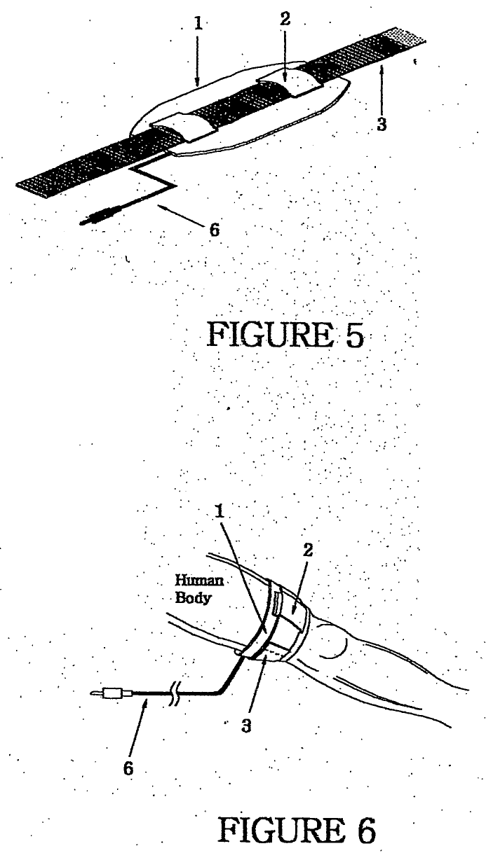

When in use, the parts are arranged, assembled and all strapped together as shown in FIG. 5 and are finally applied directly to that part of the body where the herbs are to be administered, for example, to the upper arm of the person, as shown in FIG. 6.

The present invention has been described in detail. However it is to be understood that the detailed description and the specific examples, while indicating preferred embodiments of the invention, are given, by way of illustration only, since various changes and modifications within the gambit and scope of the invention will become apparent to those skilled in the art from this detailed description.

1. A herbal applicator for the application of herbs that is to be heated and applied directly on to the skin of the body comprises of a flexible Main Housing which can be easily attached on to the body, is affixed with a flexible Heater Pad that connects to an electrical Power Source to be regulated by an adjustable Temperature Controller characterized in that the said flexible Heater Pad rests on a Herbal Pack which is in turn retained by a flexible Herbal Pack Retainer made of a permeable material and built in such a way as to allow, easy removal of the Herbal Pack for replenishment, wherein when the herbs inside the Herbal Pack are heated, until they vaporize the Herbal Pack Retainer then will allow the vapourized herbal essence to permeate through it and onto the skin and at the same time absorb part of the heat and helps to reduce the vapourizing temperature to a curie temperature

2. A herbal applicator as claimed in claim 1 wherein the flexible main housing is made up of two sections, a top member where the strap means is located and a bottom member where the flexible heater pad is located, such that the top member helps to prevent the heat generated from the heater pad from being dispersed to the outside, whereas the bottom member helps to contain the heat generated by the heater pad within to heat the herbs effectively.

3. A herbal applicator as claimed in claim 1 wherein the heater pad is affixed with an attachment means and a corresponding attachment means is affixed on to the herbal pad retainer, the said attachment means is to facilitate easy removal and attaching of the herbal pad.

4. A herbal applicator as claimed in claim 3, wherein the attachment means is a set of velcro tape.

5. A herbal applicator as claimed in claim 1, wherein the electrical source is a portable battery.

6. A herbal applicator as claimed in claim 1, wherein the temperature control is for regulating the required temperature of the Heater Pad in order for it to vapourize the herbs contained in the Herbal Pack.

7. A herbal applicator as claimed in, claim 1 wherein the flexible Main Housing is made of a flexible material, that easily adapts to the contours of the body.

8. A method of administering herbs on to the skin of the body by heating the herbs until they vapourize, is by using a devise that comprises of a flexible Main Housing, which can be easily attached on to the body, and affixed with a flexible Heater Pad that is connected to an electrical Power Source to be regulated by all adjustable Temperature Controller, with the said flexible Heater Pad resting on a Herbal Pack which is in turn retained by a flexible Herbal Pack Retainer made of a permeable material and built in such a way as to allow easy removal of the Herbal Pack for replenishment, wherein when the herbs inside the Herbal Pack are heated, until they vaporize, the Herbal Pack Retainer then will allow the vapourized herbal essence, to permeate through it and onto the skin and at the same time absorb part of the heat and helps to reduce the vapourizing temperature to a curie temperature.
